(19) United States
(12) Patent Application Publication
(10) Pub. No.: US 2023/0321024 A1
(43) Pub. Date: Oct. 12, 2023

(54) METHODS OF TREATING VISUAL DISORDERS USING DAILY LOW DOSING OF A RETINOID COMPOUND

(71) Applicant: ELUMINEX BIOSCIENCES (SUZHOU) LIMITED, Suzhou (CH)

(72) Inventors: Glenn NORONHA, La Jolla, CA (US); Nathan TEUSCHER, Trophy Club, TX (US); Barrett KATZ, Cutchogue, NY (US)

(21) Appl. No.: 17/998,370

(22) PCT Filed: Jun. 8, 2021
(86) PCT No.: PCT/US2021/036297

§ 371 Date: Nov. 10, 2022

Related U.S. Application Data
(60) Provisional application No. 63/036,862, filed on Jun. 9, 2020.

(51) Int. Cl. A61K 31/232; A61P 27/02

(52) U.S. Cl. CPC A61K 31/232; A61P 27/02

(57) ABSTRACT

Provided herein are methods of treating a subject having a visual disorder comprising administering daily to the subject, a dosage of about 0.1 mg to 20 mg of a retinoid compound. In some embodiments, the retinoid compound is 9-cis-retinyl acetate.

### CROSS-REFERENCES TO RELATED APPLICATIONS

This application claims the benefit of priority under 35 U.S.C. § 119(e) to U.S. Provisional Application Ser. No. 63/036,862 filed Jun. 9, 2020, the disclosure of which is incorporated herein by reference in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, ORA COMPUTER PROGRAM LISTING APPENDIX SUBMITTED ON A COMPACT DISK

NOT APPLICABLE

### BACKGROUND OF THE INVENTION

Some types of inherited retinal deficiencies disrupt or interfere with the production, conversion and/or regeneration of 11-cis-retinal, which is a key vitamin A derivative in the retinoid or visual cycle. 11-Cis-retinal is an endogenous retinoid produced in and by the retinal pigment epithelium (RPE) from the isomerization and oxidation of all-trans-retinol (vitamin A derived from the diet). 11-Cis-retinal functions as a chromophore and binds covalently and reversibly to the protein opsin to form rhodopsin. Vision is initiated when a light photon is captured by 11-cis-retinal, resulting in the isomerization to all-trans-retinal and dissociation from opsin. For vision to be sustained, the cycling of all-trans-retinal back into 11-cis-retinal, which occurs by a complex series of biochemical reactions (reduction of the aldehyde to the all-trans-retinol; esterification of the alcohol; simultaneous isomerization of trans to cis and hydrolysis to 11-cis-retinol; and oxidation to 11-cis-retinal) involving multiple enzymes and proteins in the retinoid or visual cycle, is required.

Endogenous retinoid deficiencies, such as those caused by mutations in the genes encoding the enzymes and proteins utilized in the visual cycle or those caused by the aging process, impair the synthesis or regeneration of 11-cis-retinal, the result of which leads to progressive loss of visual function and eventually to blindness due to the shortage or depletion of 11-cis-retinal due to an inability to transduce photo-signals required for vision.

LRAT and RPE65 are genes that are both critical for the visual cycle. The LRAT gene encodes the enzyme lecithin:retinol acetyltransferase (LRAT) and the RPE65 gene encodes the protein retinal pigment epithelial protein 65 (RPE65). The enzyme LRAT is responsible for esterification of 11-trans-retinol in the visual cycle while RPE65 simultaneously hydrolyzes the 11-trans-retinol ester to the alcohol and isomerizes it so the resulting product is 11-cis-retinol. An oxidation step then results in 11-cis-retinal. Each of the enzymes, LRAT and RPE65, is therefore responsible for a critical step in the regeneration of 11-cis-retinal.

Phenotypically, mutations in either LRAT or RPE65 were diagnosed as sub-types of Leber congenital amaurosis (LCA) or as subtypes of Retinitis pigmentosa (RP). LCA is a cause of inherited childhood blindness that affects children from birth or shortly thereafter. Patients with LCA lack the ability to generate 11-cis-retinal in adequate quantities and therefore suffer from severe vision loss at birth, nystagmus, poor pupillary responses and severely diminished electroretinograms (ERGs). Mutations in the LRAT or RPE65 genes are also associated with autosomal recessive retinitis pigmentosa (arRP), which is a subset of hereditary retinitis pigmentosa (RP) which is characterized by degeneration of rod and cone photoreceptors. Patients with arRP may lose vision either in childhood or in mid-life. The classic pattern of vision loss includes difficulties with dark adaptation and night blindness in adolescence and loss of mid-peripheral visual field in young adulthood. arRP typically presents itself as primary rod degeneration with secondary degeneration of cones and is thus described as a rod-cone dystrophy, with rods being more affected than cones. This sequence of the photoreceptor cells involvement explains why arRP patients initially exhibit night blindness, and only in later life become visually impaired in diurnal conditions (Hamel C., Orphanet Journal of Rare Diseases L40 (2006)). arRP is the diagnosis given to patients with photoreceptor degeneration who have good central vision within the first decade of life, although arRP onset can also occur much later at either the beginning of mid-life or after mid-life (“late onset arRP”). As the disease progresses, patients lose far peripheral vision, eventually develop tunnel vision, and finally lose central vision by the age of 60 years.

Retinitis Punctata Albesciens is another form of Retinitis Pigmentosa that exhibits a shortage of 11-cis-retinal in the rods. Aging also leads to the decrease in night vision and loss of contrast sensitivity due to a shorting of 11-cis retinal. Excess unbound opsin is believed to randomly excite the visual transduction system. This can create noise in the system, and thus more light and more contrast is necessary to see well.

Congenital Stationary Night Blindness (CSNB) and Fundus Albipunctatus are a group of diseases that are manifested as night blindness, but there is not a progressive loss of vision as in the case of RP. Some forms of CSNB are due to a delay in the recycling of 11-cis-retinal. Fundus Albipunctatus until recently was thought to be a special case of CSNB where the retinal appearance is abnormal with hundreds of small white dots appearing in the retina. It has been shown recently that this is also a progressive disease although much slower than Retinitis Pigmentosa. It is caused by a gene defect that leads to a delay in the cycling of 11-cis-retinal.

Endogenous retinoid deficiencies can also be associated with the aging process, even in the absence of inherited gene mutations of the genes encoding the enzymes and proteins utilized in the visual cycle. Age-related visual disorders include, for example, loss of night vision, nyctalopia and contrast sensitivity due to a shortage of 11-cis-retinal. This is consistent with the finding that a dramatic slowing of rod-mediated dark adaptation after light exposure associated with human aging is related to a delayed regeneration of rhodopsin (Jackson, G. R. et al,. J. Vision Research 39, 3975-3982 (1999)). In addition, excess unbound opsin (due to 11-cis-retinal shortage) is believed to randomly excite the visual transduction system. This can create noise in the system, and thus necessitates more light and/or more contrast in order to see well.

Animal models have shown that retinoid compounds which are highly-light sensitive compounds are photoisomerized or “bleached” by light from the retina within just a few hours unless the eyes are covered. These studies were conducted with animals kept in the dark during and following treatment with synthetic retinoids until the evaluation period in order to minimize photoisomerization/bleaching of the synthetic retinoid. Batten ML et al. “Pharmacological and rAAV Gene Therapy Rescue of Visual Functions in a Blind Mouse Model of Leber Congenital Amaurosis” PLo-S Medicine 2005; 2:333; Margaron, P., Castaner, L., and Narfstrom, K. “Evaluation of Intravitreal cis-Retinoid Replacement Therapy in a Canine Model Of Leber's Congenital Amaurosis” Invest Ophthalmol Vis Sci 2009; 50:E-Abstract 6280; Gearhart P M, Gearhart C, Thompson D A, Petersen-Jones S M. “Improvement of visual performance with intravitreal administration of 9-cis-retinal in Rpe65-mutant dogs” Arch Ophthalmol 2010; 128(11): 1442-8.

Frequent administration of any retinoid to compensate for the bleaching effect implicates the well-known toxicity of the retinoid class of the compounds. See, Teelmann, K “Retinoids: Toxicity and Teratogenicity to Date,” Pharmac. Ther., Vol. 40, pp 29-43 (1989); Gerber, L E et al “Changes in Lipid Metabolism During Retinoid Administration” J. Amer. Acad. Derm., Vol. 6, pp 664-74 (1982); Allen L H “Estimating the Potential for Vit A Toxicity in Women and Young Children” J. Nutr., Vol. 132, pp. 2907-19 (2002); Silverman, AK “Hypervitaminosis A Syndrome: A Paradigm of Retinoid Side Effects”, J. Am. Acad. Derm., Vol. 16, pp 1027-39 (1987); Zech L A et al. “Changes in Plasma Cholesterol and Triglyceride Levels After Treatment with Oral Isotretinoin” Arch. Dermatol., Vol. 119, pp 987-93 (1983). Toxicity caused by chronic administration of retinoids can cause changes in lipid metabolism, damage to the liver, nausea, vomiting, blurred vision, damage to bones, interference with bone development and several other serious undesirable effects.

In the context of treating the loss or impairment of vision due to retinoid deficiency, which is a chronic condition requiring lifetime treatments, these toxic effects can be very important and require careful thought and consideration. Moreover, the negative side effects are of particular concern in young patients, whose susceptibility to side effects related to their physical development is well documented.

This combination of a need for repeated administration in response to bleaching, and the undesirable serious side effects of repeated administration, poses a problem for the use of synthetic retinoids to treat the loss of vision caused by retinoid deficiency. Earlier work evaluated the usefulness of retinoids as a treatment for these disorders and concluded that retinoids and similar compounds are simply not good clinical candidates for the treatment of retinoid deficiency disorders. See, Fan J. et al. “Light Prevents Exogenous 11-cis Retinal from Maintaining Cone Photoreceptors in Chromophore-deficient Mice”, Invest. Ophthalmol.Vis Sci. Jan. 12, 2011, 10-6437.

Previous work aiming to compensate for the known negative effects of retinoid compound administration developed dosing schemes that include a set period for retinoid compound administration followed by a required drug holiday or “resting period” (time without drug administration) have been developed (see, WO2011/13208 and WO2013/134867). Notably, in recognition of the negative side effects posted by retinoid compounds when higher doses are administered, each of these dosing schemes expressly avoid prolonged daily dosing.

Despite continued efforts to develop retinoid compounds for the treatment of visual disorders, none have been approved by the FDA or any other regulatory body. As such, there remains a need for the development of dosing schemes for retinoid compounds that can properly balance the need for improving visual function while avoiding or minimizing adverse effects to provide appropriate benefit while reducing risk for the patient.

The present disclosure addresses this need and provides related advantages as well.

### BRIEF SUMMARY OF THE INVENTION

In some aspects, provided herein are methods of treating a subject having a visual disorder comprising administering daily to the subject, a dosage of about 0.1 mg to 20 mg of a retinoid compound. In some embodiments, the retinoid compound is 9-cis-retinyl acetate.

In some embodiments, the total daily dosage of the retinoid compound is about 1 mg. In some embodiments, the total daily dosage of the retinoid compound is about 2 mg. In some embodiments, the retinoid compound is administered once daily.

In some aspects, provided herein are methods of treating a subject having a visual disorder comprising administering to the subject an effective amount of a retinyl ester once daily, wherein the effective amount of the retinyl ester maintains a trough circulating blood concentration of a corresponding retinyl alcohol of at least 2 nM.

In additional aspects, provided herein are a single unit dosages and kits having about 0.10-20 mg of 9-cis-retinyl acetate.

Other objects, features, and advantages of the present invention will be apparent to one of skill in the art from the following detailed description and figures.

### BRIEF DESCRIPTION OF THE DRAWINGS

FIG. 1 provides a schematic drawing of the retinoid cycle.

FIG. 2 plots human pharmacokinetic (PK) data for the circulating levels of 9-cis-retinol in the blood from completed clinical trials where 9-cis-retinyl acetate was administered orally.

FIG. 3 schematically illustrates a 2 compartment PK model used to describe the observed clinical data.

FIG. 4 shows observed and predicted 9-cis-retinol circulating blood concentrations from a population PK model for levels of circulating blood 9-cis-retinol up to 700 h post dose.

FIG. 5 plots the predicted 9-cis-retinol circulating blood concentrations in human patients receiving a low daily dose of 9-cis-retinyl acetate.

### DETAILED DESCRIPTION OF THE INVENTION

### I. General

The present disclosure is based, in part, on the surprising discovery that daily low dosing of a retinoid compound or a derivative thereof, without a resting period, can be used to effectively improve visual function in subjects with visual disorders caused by compromised portions of the visual cycle. Advantageously, this daily dosing regimen not only provides effective improvements in visual function, it also minimizes the well-known adverse drug reactions associated with administration of retinoids.

In particular, prior to this disclosure, it was generally believed that daily dosing would undoubtedly lead to accumulation of retinoid compound and its metabolites resulting in undue and increased incidence, severity and prolongation of adverse drug events. Recognizing these issues, prior clinical work in this area focused on dosing schedules that included drug holiday resting periods where no therapeutically active compound was administered.

Unexpectedly, the current inventors have discovered that administering a low amount of a retinoid compound on a daily dosing schedule can achieve a trough steady state drug concentration that can provide a clinical improvement in vision while also minimizing, reducing, or eliminating the undesirable side-effects associated with the administration of retinoid compounds.

### II. Definitions

The term “visual disorders” refers broadly to disorders in the photoreceptors, tissue or structures of the eye. Visual disorders include, but are not limited to, retinal degeneration, retinal dystrophy, loss of photoreceptor function, photoreceptor cell death and structural or functional abnormalities or deficiencies. Visual disorders of the disclosure are typically characterized by impaired or less than normal (including complete loss of) functional vision in a subject, which include for example, activities required for daily living; or visual function in a subject, which include, for example, poor visual acuity, low or lack of retinal sensitivity, narrow or undetectable visual fields, and the like.

The term “endogenous retinoid deficiency” refers to prolonged lower levels of endogenous retinoids as compared to the levels found in a healthy eye of a subject of the same species. In some cases, a healthy eye of a subject may experience transient shortage of 11 -cis-retinal, which leads to a brief period of blindness followed by vision recovery, while in subjects with an endogenous retinoid deficiency, the subject is deficient in its ability to reliably or rapidly regenerate the endogenous level of 11-cis-retinal, which leads to prolonged and/or pronounced 11-cis retinal deficits.

The term “9-cis-retinyl acetate” refers to (2E,4E,6Z,8E)-3,7-dimethyl-9-(2,6,6-trimethylcyclohex-1-en-1-yl)nona-2,4,6,8-tetraen-1-yl acetate (IUPAC name), having the following chemical structure:

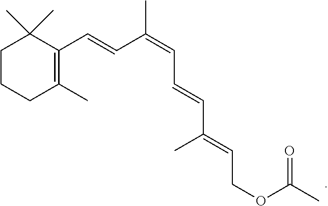 [SMILES: CC(=O)OC/C=C(C)/C=C/C=C(C)\C=C\C1=C(C)CCCC1(C)C]

### III. Detailed Description of Embodiments

### A. Methods of Treatment

Provided herein are methods of treating a subject having a visual disorder comprising administering daily to the subject, a dosage of about 0.1 mg to 20 mg of a retinoid compound.

The daily dosage of the retinoid compound is generally low and does not exceed 20 mg. In some embodiments, the daily dosage of the retinoid compound is about 0.1 mg to 20 mg, 0.25 mg to 10 mg, 0.5 mg to 5 mg, or 0.75 mg to 2.5 mg. In some embodiments, the daily dosage of the retinoid compound is about 0.1 mg to 20 mg. In some embodiments, the daily dosage of the retinoid compound is about 0.25 mg to 10 mg. In some embodiments, the daily dosage of the retinoid compound is 0.5 mg to 5 mg. In some embodiments, the daily dosage of the retinoid compound is 0.75 mg to 2.5 mg.

In some embodiments the daily dosage of the retinoid compound is about 0.1, 0.25, 0.5, 0.75, 1.0, 1.25, 1.5, 1.75, 2.0, 2.25, 2.5, 2.75, 3.0, 3.25, 3.5, 3.75, 3.0, 3.25, 3.5, 3.75, 4.0, 4.25, 4.5, 4.75, or 5 mg. In some embodiments the total daily dosage of the retinoid compound is about 0.5 mg. In some embodiments the daily dosage of the retinoid compound is about 1 mg. In some embodiments the daily dosage of the retinoid compound is about 1.5 mg. In some embodiments the total dosage of the retinoid compound is about 2 mg. In some embodiments the total dosage of the retinoid compound is about 2.5 mg. In some embodiments the total dosage of the retinoid compound is about 3 mg. In some embodiments the daily dosage of the retinoid compound is about 3.5 mg. In some embodiments the daily dosage of the retinoid compound is about 4 mg. In some embodiments the daily dosage of the retinoid compound is about 4.5 mg. In some embodiments the daily dosage of the retinoid compound is about 5 mg.

In some embodiments, the current disclosure provides methods of treating a subject having a visual disorder comprising administering daily to the subject, a dosage of about 0.1 mg to 20 mg of 9-cis-retinyl acetate. In some embodiments, the dosage of 9-cis-retinyl acetate is about 1 mg.

Typically, the daily dosage is administered in a single dose. However, dosing employing two, three or four daily administrations are also contemplated.

In some aspects, also provided herein are methods of treating a subject having a visual disorder comprising administering to the subject an effective amount of a retinyl ester once daily, wherein the effective amount of the retinyl ester maintains a trough circulating blood concentration of a corresponding retinyl alcohol of at least 2 nM.

Retinyl esters are readily deesterified (metabolized) after administration to the corresponding retinyl alcohol and other metabolites. For example, 9-cis-retinyl acetate is metabolized by de-esterification to form 9-cis-retinol. The retinyl esters described herein undergo similar reactions to form the corresponding retinyl alcohol. As described supra, the inventors of the present disclosure have surprisingly discovered that daily administration of low amounts of retinyl ester can achieve and maintain a clinically relevant trough circulating blood concertation of the corresponding retinyl alcohol (the biologically active compound that can be incorporated into the visual cycle) that improves vision and minimizes, reduces, or eliminates the undesirable side-effects associated with the administration of retinoid compounds.

In some embodiments, the effective amount of the retinyl ester maintains a trough circulating blood concentration of a corresponding retinyl alcohol of at least 1, 2, 3, 4, 5, 6 or more nM. In some embodiments, the effective amount of the retinyl ester maintains a trough circulating blood concentration of a corresponding retinyl alcohol of at least 2 nM. In some embodiments, the effective amount of the retinyl ester maintains a trough circulating blood concentration of a corresponding retinyl alcohol of at least 3 nM. In some embodiments, the effective amount of the retinyl ester maintains a trough circulating blood concentration of a corresponding retinyl alcohol of at least 4 nM.

In some embodiments, the effective amount of the retinyl ester maintains a circulating blood concentration of a corresponding retinyl alcohol from 2 nM to 20 nM. In some embodiments, the effective amount of the retinyl ester maintains a circulating blood concentration of a corresponding retinyl alcohol from 2 nM to 15 nM. In some embodiments, the effective amount of the retinyl ester maintains a circulating blood concentration of a corresponding retinyl alcohol from 2 nM to 10 nM. In some embodiments, the effective amount of the retinyl ester maintains a circulating blood concentration of a corresponding retinyl alcohol from 2 nM to 8 nM. In some embodiments, the effective amount of the retinyl ester maintains a circulating blood concentration of a corresponding retinyl alcohol from 2.5 nM to 15 nM. In some embodiments, the effective amount of the retinyl ester maintains a circulating blood concentration of a corresponding retinyl alcohol from 2.5 nM to 10 nM. In some embodiments, the effective amount of the retinyl ester maintains a circulating blood concentration of a corresponding retinyl alcohol from 2.5 nM to 8 nM. In some embodiments, the effective amount of the retinyl ester maintains a circulating blood concentration of a corresponding retinyl alcohol from 3 nM to 15 nM. In some embodiments, the effective amount of the retinyl ester maintains a circulating blood concentration of a corresponding retinyl alcohol from 3 nM to 10 nM. In some embodiments, the effective amount of the retinyl ester maintains a circulating blood concentration of a corresponding retinyl alcohol from 3 nM to 8 nM.

In some embodiments, the C. of retinyl alcohol observed after once daily dosing of retinyl ester precursor does not exceed 10, 11, 12, 13, 14, 15, 16, 17, 18, 19, 20, 21, or 22 nM. In some embodiments, the C. of retinyl alcohol observed after once daily dosing of retinyl ester precursor does not exceed 10 nM. In some embodiments, the C. of retinyl alcohol observed after once daily dosing of retinyl ester precursor does not exceed 15 nM. In some embodiments, the C. of retinyl alcohol observed after once daily dosing of retinyl ester precursor does not exceed 20 nM.

In some embodiments, provided herein are methods of treating a subject having a visual disorder comprising administering to the subject an effective amount of 9-cis-retinyl acetate once daily, wherein the effective amount of 9-cis-retinyl acetate maintains a trough circulating concentration of 9-cis-retinol of at least 2 nM.

The daily low doses of retinoid compound described herein can surprisingly be maintained for an extended period of time without the need for a disruption in administration (i.e., a drug holiday). For example, in some embodiments, the daily administration described herein can continue for 14, 21, 28, 35, 42, 49, 56, 63, 70, 77, 84, 91, 98, 105, 112, 119, 126, 133, 140 147, 154, 161, 168, 175, 182, 189, 196, 203, 210, 217, 224, 231, 238, 245, 252, 259, 266, 273, 280, 287, 294, 301, 308, 315, 322, 329, 336, 343, 350, 357, 364, or more days without the need for a holiday. In some embodiments, the daily administration described herein can continue for 15, 30, 45, 60, 75, 90, 105, 120, 135, 160, 175, 190, 205, 220, 235, 250, 265, 280, 295, 310, 325, 330, 335, 360 or more days without the need for a holiday. In some embodiments, the daily administration described herein can continue for 1, 2, 3, 4, 5, 6, 7, 8, 9 10, 11, 12, 13, 14, 15 or more years without the need for a holiday.

In some embodiments of the above described methods, the retinoid compound is administered orally.

### B. Retinoid Compounds

The present disclosure provides methods of restoring or stabilizing photoreceptor function in a subject's visual system. Synthetic retinal derivatives can be administered to restore or stabilize photoreceptor function, and/or to ameliorate the effects of a deficiency in retinoid levels. Photoreceptor function can be restored or stabilized, for example, by providing a retinoid compound that can act as an 11-cis-retinoid replacement and/or an opsin agonist. The retinoid compound can also ameliorate the effects of a retinoid deficiency on a subject's visual system. A retinoid compound can be administered prophylactically or therapeutically to a subject.

Retinoid compounds of the present disclosure include naturally occurring and synthetic compounds bearing the general structure of vitamin A (retinol) and variations on that structure which bear similarities to retinol in terms of biological activity. In some embodiments the retinoid compounds of the present disclosure are esterified prodrugs (retinyl esters) such as 9-cis-retinyl esters or 11-cis-retinyl esters. In some embodiments, the 9-cis-retinyl ester is 9-cis-retinyl acetate, 9-cis-retinyl propionate, 9-cis-retinyl butyrate, 9-cis-retinyl pentanoate, 9-cis-retinyl palmitate, 9-cis-retinyl stearate, 9-cis-retinyl oleate or the like. In some embodiments, the 11-cis-retinyl ester is 11-cis-retinyl acetate, 11-cis-retinyl propionate, 11-cis-retinyl butyrate, 11-cis-retinyl pentanoate, 11-cis-retinyl palmitate, 11-cis-retinyl stearate, 11-cis-retinyl oleate or the like.

In some embodiments the retinoid compound is 9-cis-retinyl acetate.

In some embodiments the retinoid compound is 9-cis-retinyl propionate.

In some embodiments the retinoid compound is 11-cis-retinyl acetate.

In some embodiments the retinoid compound is 11-cis-retinyl propionate.

### C. Visual Disorders

The therapeutic regimens and methods of the disclosure are for the treatment and amelioration of visual disorders. In some embodiments, the visual disorder is an endogenous retinoid deficiency. Typically, the endogenous retinoid deficiency causes loss of visual function.

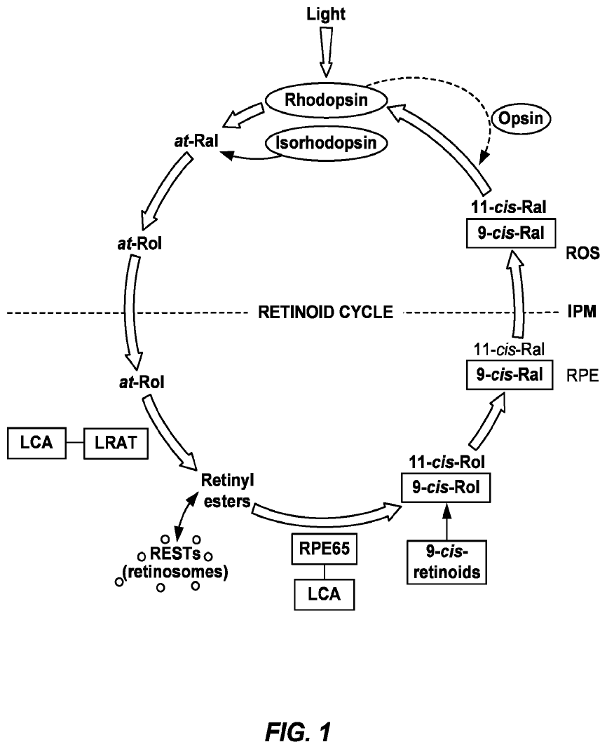

Endogenous retinoid deficiency can be caused by one or more defects in the visual cycle which includes enzymatic deficiencies and impaired transport processes between the photoreceptors and retinal pigment epithelial cells (RPE). FIG. 1 schematically shows a vertebrate, preferably the human, visual cycle (or retinoid cycle), which operates between the RPE and the outer segments of photoreceptors. 11-cis-retinal is regenerated through a series of enzymatic reactions and transport processes to and from the RPE after which it binds to opsin to form rhodopsin in the photoreceptor. Rhodopsin is then activated by light to form meta-rhodopsin which activates the phototransduction cascade while the bound cis-retinoid is isomerized to all-trans-retinal (von Lintig, J. et al., Trends Biochem Sci Feb 24 (2010)).

Mutations in more than a dozen genes encoding retinal proteins have been identified that participate in several biochemical pathways in the visual cycle. For example, mutations in genes that encode lecithin:retinoid acetyl transferase (the LRAT gene) and retinal pigment epithelium protein 65 kDa (the RPE65 gene) disrupt the retinoid cycle, resulting in a deficiency of 11-cis-retinal, an excess of free opsin, an excess of retinoid waste (e.g., degradation) products and/or intermediates in the recycling of all-trans-retinal, or the like.

Endogenous retinoid levels in a subject's eyes and deficiencies of such levels may be determined in accordance with the methods disclosed in, for example, U.S. Published Patent Application No. 2005/0159662 (the disclosure of which is incorporated by reference herein in its entirety). Other methods of determining endogenous retinoid levels in a vertebrate eye and a deficiency of such retinoids include, for example, analysis by high pressure liquid chromatography (HPLC) of retinoids in a blood sample from a subject. For example, a blood sample can be obtained from a subject and retinoid types and levels in the sample can be separated and analyzed by normal phase high pressure liquid chromatography (HPLC) (e.g., with a HP 1100 HPLC and a Beckman, Ultrasphere-Si, 4.6 mm×250 mm column using 10% ethyl acetate/90% hexane at a flow rate of 1.4 ml/minute). The retinoids can be detected by, for example, detection at 325 nm using a diode-array detector and HP Chemstation A.03.03 software. A deficiency in retinoids can be determined, for example, by comparison of the profile of retinoids in the sample with a sample from a control subject (e.g., a normal subject).

Various conditions can cause a subject to be predisposed to or develop endogenous retinoid deficiency. For example, a subject that has an RPE65 gene mutation or an LRAT gene mutation is genetically predisposed to endogenous retinoid deficiency and visual impairment that ultimately lead to complete vision loss and severe retinal dystrophy. In particular, RPE65 and LRAT gene mutations are found in both LCA and arRP patients. Even in the absence of any genetic defects in the visual cycle, an aging subject may nonetheless develop endogenous retinoid deficiency.

Exemplary of visual disorders of the current disclosure are further discussed below.

### i. Leber Congenital Amaurosis (LCA)

One visual disorder associated with endogenous retinoid deficiency is Leber Congenital Amaurosis (LCA). LCA is an inherited childhood disease with early onset vision loss and retinal dystrophy. Mutations in the RPE65 gene in patients with autosomal recessive retinitis pigmentosa (arRP) or Leber congenital amaurosis have been reported to cause 0.5% and 6% of LCA cases, respectively (den Hollander, A. I. et al., Prog Ret Eye Res 27: 391-419, (2008) and den Hollander, A. I. et al., Proc Natl Acad Sci U S A 95: 3088-93 (1998)). These forms are characterized by a significant deficiency of 11-cis-retinal, the visual chromophore that binds rod and cone opsins to form the visual pigments (rhodopsin and cone- pigments) (Redmond, T. M. et al., Nat Gen 20: 344-51 (1998) and Batten, M. L. et al., J Biol Chem 279: 10422-32 (2004)). Chronic deficiency of 11-cis-retinal eventually results in photoreceptor degeneration (Travis, G. H. et al., Annu Rev Pharmacol Toxicol 47: 469-512 (2007)). The interval between the loss of visual function and retinal degeneration creates an opportunity for vision rescue.

In subjects having LCA due to an RPE65 gene mutation, retinyl esters build up in the retinal pigment epithelium (RPE) (Thompson, D. A. et al., Nat Gen 28:1 23-4 (2001) and Gu S. M. et al., Nat Gen 17: 194-7 (1997)), which eventually results in retinal degeneration.

Subjects having LCA due to an LRAT gene mutation are unable to make esters and subsequently secrete any excess retinoids, which are associated with early-onset severe retinal dystrophy and retinal degeneration (Morimura H et al. Proc Natl Acad Sci U S A 95: 3088-93 (1998)).

### ii. Retinitis Pigmentosa and Night Blindness (Nyctalopia)

Another visual disorder associated with endogenous retinoid deficiency is night blindness caused by, for example, retinitis pigmentosa (RP) or congenital stationary night blindness (CSNB).

RP is a condition caused by defects in many different genes. To date, 19 known and 17 uncharacterized gene mutations have been identified, causing great heterogeneity in the disease (Phelan, J. K. et al., Mol Vis. 6: 116-124 (2000)). The age of onset for RP, as well as the severity of the disease, is a function of the mode of inheritance. RP may be inherited by autosomal dominant, autosomal recessive, or X-linked traits. Autsomal recessive RP (arRP) can occur in 20% of all RP cases. In recent years, mutations in the LRAT and RPE65 genes have been discovered in patients with arRP. These specific mutations are linked to defects in retinoid metabolism of the visual cycle and may result in photoreceptor degeneration (Morimura, H. et al., Proc Natl Acad Sci USA. 95(6): 3088-3093 (1998)).

As noted herein, the protein encoded by the RPE65 gene has a biochemical association with retinol binding protein and 11-cis-retinol dehydrogenase and is essential for 11-cis-retinal production (Gollapalli, D. R. et al., Biochemistry. 42(19): 5809-5818 (2003) and Redmond, T. M. et al., Nat Genet. 20(4): 344-351 (1998)). Preclinical and clinical information show that loss of the function of the RPE65 protein blocks retinoid processing after esterification of vitamin A to membrane lipids and results in loss of vision.

Early stages of typical RP are characterized by night blindness and loss of mid-peripheral visual field, reflecting primary rod impairment. As the disease progresses, patients lose far peripheral and central vision, eventually leading to blindness. Prominent clinical findings include bone spicule-shaped pigment in the retina and attenuated/abnormal electroretinogram (ERG) responses. It is speculated that the absence of RPE65 products would cause a massive, early degeneration of photoreceptors while substitution of amino acids would lead to a slower pace of degeneration (Marlhens, F. et al., Eur J Hum Genet. 6(5): 527-531 (1998)).

CSNB and fundus albipunctatus are a group of diseases that are manifested as night blindness, but there is not a progressive loss of vision as in the RP. Some forms of CSNB are due to a delay in the recycling of 11-cis -retinal. Until recently, fundus albipunctatus was thought to be a special case of CSNB where the retinal appearance is abnormal with hundreds of small white dots appearing in the retina. It has been recently been shown that fundus albipunctatus is also a progressive disease, although much slower than RP. Fundus albipunctatus is caused by a gene defect that leads to a delay in the cycling of 11-cis-retinal.

### iii. Age-Related Visual Disorders

Another condition associated with endogenous retinoid deficiency is age-related decrease in retinal photoreceptor function. As discussed herein, it has been recognized that inadequate availability and/or processing of vitamin A to the visual chromophore, 11-cis-retinal, can adversely affect vertebrate rhodopsin regeneration and visual transduction (McBee, J. K. et al., Prog Retin Eye Res 20, 469-529 (2001); Lamb, T. D. et al., Prog Retin Eye Res 23, 307-380 (2004); and Travis, G. H. et al., Annu Rev Pharmacol Toxicol (2006)). In aging, rhodopsin regeneration after light exposure is more delayed in humans and mice deprived of vitamin A due to either dietary deficiency or inadequate intestinal absorption (Lamb, T. D. et al,. J. Prog Retin Eye Res 23, 307-380 (2004)). Moreover, treatment with vitamin A and its derivatives may have beneficial effects in aging and retinal diseases such as Sorbsby's fundus dystrophy and retinitis pigmentosa (Jacobson, S. G., et al., Nat Genet 11, 27-32 (1995); and Berson, E. L., et al., Arch Ophthalmol 111, 761-772 (1993)).

Age-related visual disorders include a slowing of rod-mediated dark adaptation after light exposure, a decrease in night vision (nyctalopia), and/or a decrease in contrast sensitivity. Age-related visual disorders may also include wet or dry forms of age-related macular degeneration (AMD).

AMD is one of the specific visual disorders associated with the posterior portion of the eyeball and is the leading cause of blindness among older people. AMD results in damage to the macula, a small circular area in the center of the retina. Because the macula is the area which enables one to discern small details and to read or drive, its deterioration may bring about diminished visual acuity and even blindness. People with AMD suffer deterioration of central vision but usually retain peripheral sight. In AMD, vision loss occurs when complications late in the disease either cause new blood vessels to grow under the retina or the retina atrophies.

### iv. Subject Populations

While any subject having a visual disorder associated with an endogenous retinoid deficiency (as defined herein) may be treated by the therapeutic regimens and methods of the invention, there is a physiological window of opportunity wherein the therapeutic regimen or method is the most effective in restoring visual function to the subject. Preferably, the window of opportunity for the therapeutic regimens of the invention to be the most effective in a subject is defined as the interval between loss of visual function and retinal degeneration, particularly with respect to photoreceptor cell degeneration. Subjects in certain age groups may particularly benefit from the therapeutic regimens of the invention. More specifically, subjects with a lesser degree of retinal/photoreceptor degeneration tend to have a better or faster response to the therapeutic regimen of the invention and/or may have a longer resting period before a subsequent dosing period is needed.

For example, in certain embodiments, younger subjects with a loss of visual function due to LCA or RP may retain a higher percentage of dormant photoreceptors. Such dormant photoreceptors are capable of responding to the therapeutic regimens of the invention. In particular, in treating loss of visual function in a subject arising from inherited childhood blindness such as LCA or early onset RP, such as arRP, younger subjects may expect a greater recovery of visual functions because their retinal degeneration is less advanced. Thus, in one embodiment of the invention, the subject is a human juvenile, i.e., younger than 15 years, old upon commencement of the therapeutic regimen. In other embodiments of the invention, the subject is a human newborn or a human infant younger than 1 year old, younger than 18 months, younger than 24 months or younger than 36 months old when the therapeutic regimen is commenced. In other embodiments, the subject is a human of 5 years old or older when the therapeutic regimen is commenced. In further embodiments, the human subject is 10 years old or older when the therapeutic regimen is commenced.

In some instances, RP may appear in a human subject during the second decade or even later. The average age of diagnosis for arRP in a human is about 36 years old (Tsujikawa M. et al., Arch Ophthalmol 126(3) 337-340 (2008)). Thus, in other embodiments, the human subject is 15 years old or older when the therapeutic regimen is commenced. In more specific embodiments, the human subject is 20 years old or older, 30 years old or older, 40 years or older, 50 years or older, 60 years or older or 70 years or older when the therapeutic regimen is commenced.

In further embodiments, the human subject is an aging subject suffering from age-related retinal disorders. As used herein, an aging human subject is typically at least 45, or at least 50, or at least 60, or at least 65 years old when the therapeutic regimen is commenced.

Preferably, for any of these subjects, the therapeutic regimens and methods of the invention should commence as soon as a diagnosis of a visual disorder as defined herein is ascertained, such that any degeneration of the retina, in particular the photoreceptors, has not reached a point where the therapeutic regimens of the invention would be ineffective in treating or ameliorating the visual disorder in the subject.

### D. Unit Dosage Forms & Kits

The methods described herein use low daily dosing to treat various visual disorders. Daily dosing is best achieved through oral administration of the retinoid compounds, particularly 9-cis-retinyl acetate. Thus, provided herein are single unit dosage forms and kits of 9-cis-retinyl acetate.

The dosage form may be in any form suitable for oral administration, including, but not limited to, a capsule or a liquid enclosed within a vial, a syringe, an ampoule other container-closure system approved by the Food and Drug Administration (FDA) or other regulatory body, which may provide one or more unit dosages containing 9-cis-retinyl acetate. The kits may contain unit daily doses with approximately 1, 2, 3 or 4 weeks supply, or more.

In some embodiments, the present disclosure provides a single unit dosage capsule containing 0.1-20 mg of 9-cis-retinyl acetate.

In some embodiments, the amount of 9-cis-retinyl acetate is from about 0.25 mg to 10 mg. In some embodiments, the amount of 9-cis-retinyl acetate is from about 0.5 mg to 5 mg. In some embodiments, the amount of 9-cis-retinyl acetate is from about 0.75 mg to 2.5 mg. In some embodiments, the amount of 9-cis-retinyl acetate is about 0.5 mg. In some embodiments, the amount of 9-cis-retinyl acetate is about 1 mg. In some embodiments, the amount of 9-cis-retinyl acetate is about 2 mg.

In some embodiments, the single unit dosage form of 9-cis-retinyl acetate is a capsule.

In some embodiments, the single unit dosage form of 9-cis-retinyl acetate is a liquid enclosed within a vial, a syringe, or an ampoule.

In some embodiments, the single unit dosage form is in a capsule of size #000, #00, #0, #1, #2, #3, #4, or #5. In some embodiments, the single unit dosage form is in a capsule of size #000. In some embodiments, the single unit dosage form is in a capsule of size #00. In some embodiments, the single unit dosage form is in a capsule of size #0. In some embodiments, the single unit dosage form is in a capsule of size #1. In some embodiments, the single unit dosage form is in a capsule of size #2. In some embodiments, the single unit dosage form is in a capsule of size #3. In some embodiments, the single unit dosage form is in a capsule of size #4. In some embodiments, the single unit dosage form is in a capsule of size #5.

The single unit dosage form of 9-cis-retinyl acetate or the other retinoid compounds described herein can be formulated in a liquid delivery vehicle that optionally further includes an antioxidant. In some embodiments, the liquid delivery vehicle is an oil. In some embodiments, the liquid delivery vehicle is soybean oil. In some embodiments, the soybean oil is a U.S.P. grade soybean oil. In some embodiments, the antioxidant is butylated hydroxyanisole (BHA). The concentration of antioxidant can include 0.05%, 0.1%, 0.15%, 0.2% or other suitable amounts.

The disclosure also encompasses kits comprising dosage forms of the current disclosure.

In some aspects, the present disclosure provides a kit that includes 9-cis-retinyl acetate. Some of the kits described herein include a label describing a method of administering 9-cis-retinyl acetate as described herein. Some of the kits described herein include a label and additional instructions describing a method of treating Leber congenital amaurosis (LCA) by administering daily to the subject, a dosage of about 0.1 mg to 20 mg of 9-cis-retinyl acetate or a sub-embodiment described herein. In some embodiments, the kits described herein include a label and additional instructions describing a method of treating retinitis pigmentosa (RP) by administering daily to the subject, a dosage of about 0.1 mg to 20 mg of 9-cis-retinyl acetate or a sub-embodiment described herein. In some embodiments, the kits described herein include a label describing a method of treating an endogenous retinoid deficiency by administering daily to the subject, a dosage of about 0.1 mg to 20 mg of 9-cis-retinyl acetate or a sub-embodiment described herein. In some embodiments, the kits described herein include a label describing a method of treating age-related macular degeneration (AMD) by administering daily to the subject, a dosage of about 0.1 mg to 20 mg of 9-cis-retinyl acetate or a sub-embodiment described herein.

The unit dosage forms of the present invention, can be stored in a bottle, jar, vial, ampoule, tube, blister pack, or other container-closure system approved by the Food and Drug Administration (FDA) or other regulatory body, which may provide one or more unit dosages described herein. The package or dispenser may also be accompanied by a notice associated with the container in a form prescribed by a governmental agency regulating the manufacture, use, or sale of pharmaceuticals, the notice indicating approval by the agency. In certain aspects, the kit may include a formulation or composition as described herein, a container closure system including the formulation or one or more dosage units form including the formulation, and a notice or instructions describing a method of use as described herein.

### E. Evaluation of Therapeutic Effect

The therapeutic effect for patients receiving the described treatments can be determined using various techniques known in the art. These techniques include those described in

WO2011/132084 and WO2013/134867, the contents of which are hereby incorporated by reference for all purposes. These techniques also include, but are not limited to, visual navigational challenge (VNC) testing at various luminance levels, Visual Field (VF) evaluation, low luminance low contrast (LLLC), best corrected visual acuity (BCVA), high luminance high contrast (HLHC), BCVA, optical coherence tomography (OCT), and patient reported outcome (PRO) quality of life (QoL) questionnaires including a low luminance (LL) questionnaire. These techniques are validated and established assessments of visual function. A person of skill in the art understands how to perform and evaluate the results of the above-referenced assessments.

VNC testing, for example, is a visual assessment performed that has subjects complete a pre-set course with obstacles and navigational demarcations (e.g. arrows for turns) at various luminance levels. The VNC test is from Ora, Inc. The VNC tests will be adjudicated and evaluated in a similar manner, will have outcomes that will be similarly evaluated, and will be considered in a similar manner as a measure of functional vision by the US FDA and other regulatory bodies. Exemplary tested luminance levels are shown in Table 1, below

TABLE 1
Lux luminance Level

| | | | | | | | | |
|---|---|---|---|---|---|---|---|---|
| Luminance level (lux) | 0.35 | 1 | 3 | 8 | 22 | 63 | 178 | 500 |

In some embodiments, administration of 9-cis-retinyl acetate using the methods described herein results in an improvement of the VNC score of a subject relative to a baseline score prior of the subject prior to administration of 9-cis-retinyl acetate. In some embodiments, the VNC score of the subject improves by at least 1 luminance level relative to the baseline VNC score of the subject prior to administration of 9-cis-retinyl acetate. For example, in some embodiments, a subject is able to complete the VNC course at an 8 lux luminance level, where prior to treatment they could only complete the VNC course at the 22 lux luminance level. In some embodiments, the VNC score of the subject improves by at least 2 luminance levels relative to the baseline VNC score of the subject prior to administration of 9-cis-retinyl acetate. In some embodiments, the VNC score of the subject improves by at least 3 luminance levels relative to the baseline VNC score of the subject prior to administration of 9-cis-retinyl acetate. In some embodiments, the VNC score of the subject improves by at least 4 luminance levels relative to the baseline VNC score of the subject prior to administration of 9-cis-retinyl acetate. It is understood that the VNC test can be performed any time after starting treatment (e.g. after 1, 2 ,3 ,4, 5, 6, 7, 8, 9, 10, 11, 12, 13, 14, 15, 16, 17, 18, 19, 20, 21, 22, 23, 24, 25 or more weeks after starting treatment). In some embodiments, the VNC test is performed after 6 weeks of treatment. In some embodiments, the VNC test is performed after 12 weeks of treatment.

Being able to successfully complete the VNC course at a luminance level lower (“improvement”) than what was registered prior to treatment implies an ability to function at a lower level of lighting (luminance). In other words, the subject can now visually function in a darker setting, as evidenced by the ability to navigate a course that is designed to measure this ability. An improvement in 2 luminance levels shows that this improved navigation (“mobility”) is occurring at a lighting level that is a log unit (10X) lower than what was achieved prior to treatment.

In some embodiments, a patient-reported outcome (PRO) quality of life visual function questionnaire is based on the questionnaire developed by Owsley, et al. Invest Ophthalmol Vis Sci. 2006. 47(2): 528-35. As described in the referenced report, response scales are on a five-point scale with an additional option for “not applicable” if the question does not pertain to a particular subject. Questions require subjects to assess and report their answers on a difficulty scale or a frequency scale (e.g., do you have difficulty in bright sunlight?: (1) no difficulty at all, (2) a little difficulty, (3) some difficulty, (4) a lot of difficulty, (5) completely blind under these conditions, (5) stopped doing this activity because of my vision; Do you depend on others to help you because of your vision at night or under poor lighting?: (1) none of the time, (2) a little of the time, (3) some of the time, (4) most of the time, (5) completely blind under these conditions, (5) stopped doing this because of vision). The questionnaire is divided into six subscales: driving, extreme lighting, mobility, emotional distress, general dim lighting, and peripheral vision.

In some embodiments, the methods provided herein improve, stabilize or delay worsening in at least one subscale of the PRO QoL questionnaire relative to a baseline score of the subject prior to administration of 9-cis-retinyl acetate. In some embodiments, administration of 9-cis-retinyl acetate results in an improvement of at least one subscale score of the PRO QoL questionnaire relative to a baseline score of the subject prior to administration of 9-cis-retinyl acetate. For example, in some embodiments, administration of 9-cis-retinyl acetate results in an improvement in one or more subscale (e.g., the driving subscale, the extreme lighting subscale, the mobility subscale, the emotional distress subscale, the general dim lighting subscale, or the peripheral vision subscale) relative to a baseline score of the subject prior to administration of 9-cis-retinyl acetate.

### IV. EXAMPLES

The following examples are offered to illustrate, but not to limit, the claimed invention.

### Example 1: Pharmacokinetic Modeling of 9-cis-retinyl Acetate

9-cis-retinyl acetate is the acetate ester of 9-cis-retinol. Acetate esters like 9-cis-retinyl acetate are generally short-lived because they are readily hydrolyzed either at physiological pH or by esterases in the blood and other parts of the body. Evaluation of levels of 9-cis-retinyl acetate from human blood samples following oral dosing show that 9-cis-retinyl acetate is extremely short lived and rapidly hydrolyzed to 9-cis-retinol.

Our review of PK data from clinical studies from which blood samples were collected from normal volunteers and patients with visual disorders indicated that there are several metabolites observed including polar and nonpolar moieties. Further evaluation of these PK data show that the polar and non-polar metabolites include 9-cis-retinol, and the fatty acid esters, 9-cis-retinyl linoleate, 9-cis-retinyl oleate, 9-cis-retinyl stearate, and 9-cis-retinyl palmitate. Through the PK analysis, it was identified that all the fatty acid esters exhibit an apparent equilibrium with 9-cis-retinol. Thus, further discussion focuses on 9-cis-retinol.

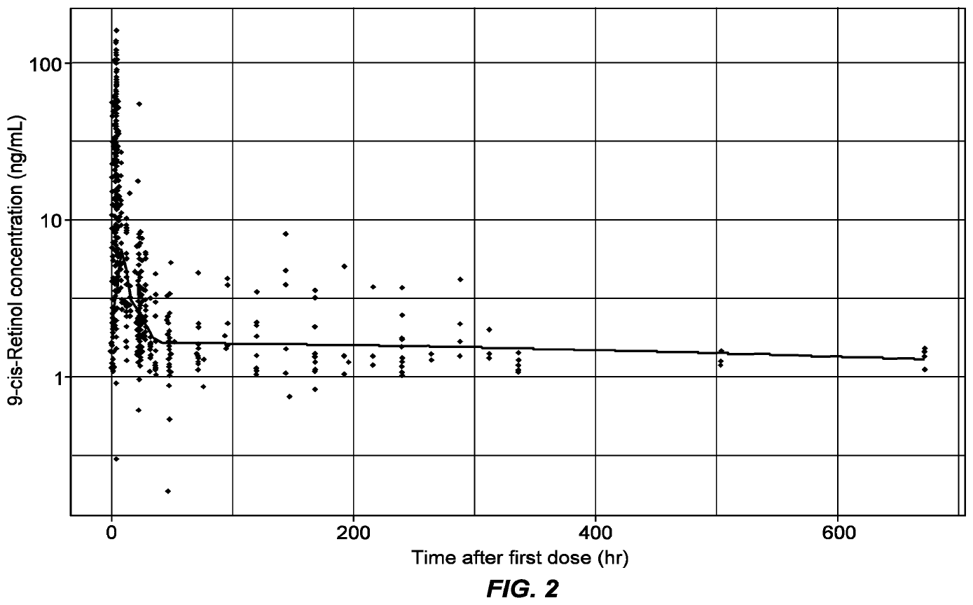

Collated in FIG. 2 are human PK data that show a Cₘₐₓ between 4 and 7 hours for 9-cis-retinol independent of initial dose administered. A low steady level of 9-cis-retinol is achieved in ˜24 hours. Surprisingly, our analysis shows that this low stead level of 9-cis-retinol persists for long periods of time after initial dosing has ceased, and it is also irrespective of initial dose administered. The circulating 9-cis-retinol is observed at similar levels from about 24 hours through at least 700 hours after a single dose.

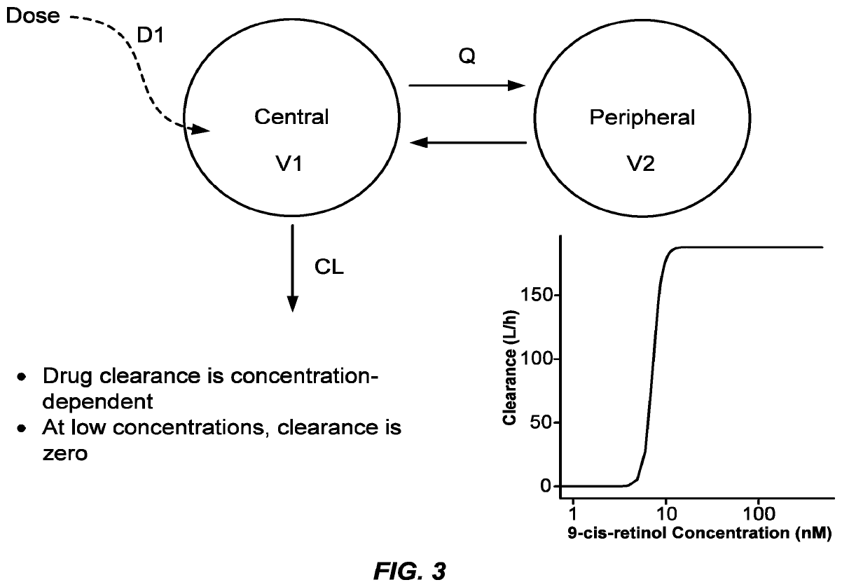

From the observed PK data analyzed, a population PK model for levels of 9-cis-retinol was built. Based on the observed clinical data, a 2 compartment PK model was constructed (FIG. 3). This kinetic model for 9-cis-retinol includes a recirculation feature that maintains 9-cis-retinol levels for long periods of time. Two features of this model include apparent concentration-dependent clearance of 9-cis-retinol from the blood, and apparent zero or close to zero clearance at low concentrations. This accounts for long recirculating levels of 9-cis-retinol, seen in samples consistently at 100 hours, 300 hours and 700 hours post last dose administered.

The clearance-concentration model plot at the bottom right of FIG. 3 shows how below a certain concentration, about 4 to 8 nmol/mL (1 to 2 ng/mL), clearance is zero, resulting in a consistent recirculation of the active 9-cis-retinol that has the potential to be a part of the visual cycle.

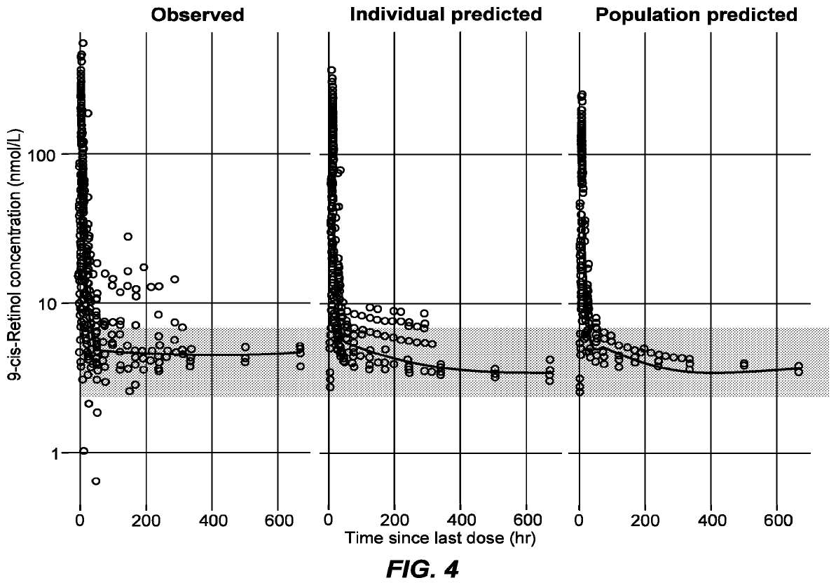

FIG. 4 shows observed and predicted data from a population PK model for levels of circulating 9-cis-retinol up to 700 h post dose using the above-mentioned model. These clinical observations and data surprisingly suggest that the body retains 9-cis-retinol for use in the visual cycle as if it were 11-cis-retinol. Clearance from the blood is rapid above ˜8 nM, but quickly drops to zero or near zero below that level. Steady state levels (from ˜24 hrs post dose) are 4.57 nM (10 mg/m² dose, 83 samples), 8.32 nM (40 mg/m² dose, 149 samples), and 7.46 nM (5 to 60 mg/m², 629 samples).

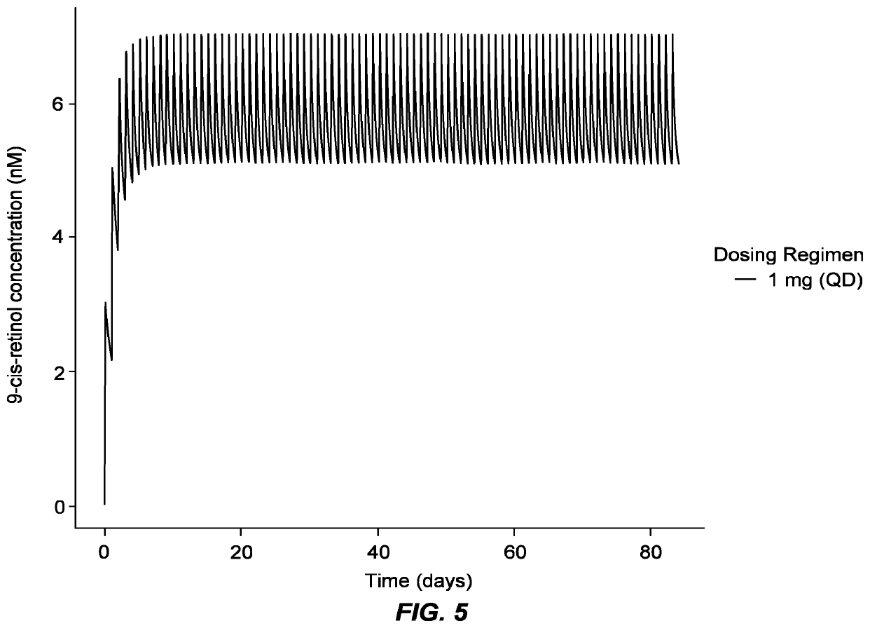

From this surprising discovery and using the constructed population PK model, a plot was prepared to visualize daily dosing of a low amount of 9-cis-retinyl acetate (1 mg) is human subjects (FIG. 5).

Based on the unexpected discovery of the very low clearance rate of 9-cis-retinol at low circulating blood concentrations and the maintaining of a tough blood concentration over time, a Phase 2/3 trial was designed. This trial is explained in further detail in Example 2.

### Example 2: Phase 2 Trial Using Low Daily Oral Dosing of a Retinyl Ester for the Treatment of Visual Disorders

### Study Objective

To evaluate the safety, efficacy and pharmacokinetics of 9-cis-retinyl acetate oral solution in subjects with IRD phenotypically diagnosed as Leber congenital amaurosis (LCA) or retinitis pigmentosa (RP) caused by RPE65 or LRAT gene mutations.

### Study Design

This is a multicenter, randomized, placebo-controlled, double-masked study in approximately 15 subjects per treatment group aged 6 years and above with IRDs, phenotypically diagnosed as LCA or RP, caused by pathologic autosomal recessive mutation in RPE65 or LRAT. Subjects either receive 0 mg of 9-cis-retinyl acetate daily (placebo control arm) or are administered 1 mg of 9-cis-retinyl acetate once daily. The study will be double masked through the week 12 visit. Subjects will be maintained on their assigned dosing schedule through the entire study.

Subjects will undergo screening evaluations during the screening period (6 weeks) before receiving study treatment (Day −42 to Day −1) including (1) on the visual navigation challenge (VNC) course to determine the luminance level at which the subject can complete and pass the course using both eyes together; (2) visual field (VF) assessments for each eye; (3) best corrected visual acuity testing for each eye; (4) optical coherence tomography (OCT) testing; and (5) genotyping is required if the subject has not had genotyping performed and documentation is not available from a certified lab.

Following randomization, subjects will be evaluated for efficacy every 4 weeks. Safety will be assessed through the entire trial at all visits. The primary efficacy analysis will be performed after all available subjects have completed their week 12 visit.

Efficacy will be assessed primarily by VNC testing at various luminance levels. Other key measurements will include VFs, low luminance low contrast (LLLC) best corrected visual acuity (BCVA), high luminance high contrast (HLHC) BCVA, optical coherence tomography (OCT), and patient reported outcome (PRO) quality of life (QoL) questionnaires including a low luminance (LL) questionnaire. Safety will be assessed systemically by vital signs, electrocardiogram (ECG), physical examination, clinical laboratory tests, bone density measurement, hand X-ray and height measurement to assess bone development, adverse events (AEs), and concomitant medications. Ocular safety will be assessed by HLHC BCVA, biomicroscopic examination, IOP, fundus examination and photography, OCT and by an evaluation of all treatment emergent (TE) AEs and serious AEs (SAEs).

### Inclusion Criteria

The following are inclusion criteria for the study:
Be aged 6 years or older.

Have a diagnosis of IRD phenotypically diagnosed as LCA or RP by an ocular geneticist or ophthalmologist and caused by pathologic biallelic autosomal recessive mutation in RPE65 or LRAT as determined by a fully accredited certified central genotyping laboratory.
Pass the VNC course tested for at least one eye designated as the study eye at a level at or above 3 lux but fail at levels below that luminance level. Both eyes will be individually evaluated, as well as both eyes together (binocular) on the VNC course. If the subject is unable to pass the VNC course at or above 3 lux binocularly, they may be included if visual acuity is 20/800 or better in at least 1 eye.
Be naïve to gene therapy, surgical implantation of prosthetic retinal chips, or subretinal injections.
If previously administered ZA as part of a clinical study, have at least >3 years since last administration of ZA.
Have a defined and recognizable retina of at least 100 microns as documented on SD-OCT.
Pregnancy testing and contraception before study treatment: Women of childbearing potential must not be pregnant or lactating. Female subjects with regular menstruation must have negative pregnancy tests at Screening (i.e., serum pregnancy test with sensitivity of ≥25 mIU/mL≥19 days before Day −1 and urine pregnancy test with sensitivity >50 mIU/mL on Day −1). Female subjects with irregular menstruation, amenorrhea, or who are taking contraception that precludes withdrawal bleeding must have a negative pregnancy test with parameters described above both at Screening and 1 month after initiating treatment with 2 approved contraceptive methods and must have been practicing 2 adequate methods of birth control for at least 1 month or complete abstinence for at least 2 months before randomization. Adequate methods of birth control include (1) use of oral contraceptives, implantable or injectable contraceptives, or an intrauterine device, with an additional barrier method (diaphragm with spermicidal gel OR condoms with spermicide OR cervical caps with spermicide); (2) a double-barrier method (diaphragm with spermicidal gel AND condoms with spermicide); (3) partner vasectomy status post 3 months or greater; and (4) total abstinence. Women who are considered postmenopausal or have undergone tubal ligation should have had their last menstrual period greater than 1 year before, OR have follicle-stimulating hormone level in the menopausal range.
Pregnancy testing and contraception during the study:

Women of childbearing potential must be willing to receive contraceptive counseling.

If the subject is female and aged <18 years, the legal guardian(s) must agree with the use of contraception.

Women of childbearing potential must practice 2 adequate methods of birth control (as described above) or complete abstinence during the treatment phase of the study and continue for 3 months after finishing the last dose of study drug.

Male subjects must either (1) not be sexually active, or (2) agree to completely abstain from sexual intercourse, or (3) have had a vasectomy with documented infertility, or (4) use a barrier method (condoms) with spermicide during sexual intercourse, during the treatment phase of the study and for an additional 3 months after finishing the last dose of study drug.
The subject or guardian who is signing the ICF understands the study procedures and agreement to participate in the study by giving written informed consent.
Be willing and able to comply with the protocol.
Be willing to adhere to the travel schedule for mobility testing on 3 occasions in approximately 3 months—that is, at the Screening/Randomization (Visit 1/2), Week 4 (Visit 3), and Week 12 (primary outcome; Visit 5); this will only apply to those subjects at sites where mobility testing is not available. Such travel will be required for 3 of the 5 total required visits. All other evaluations will be performed at the site at which the subject is enrolled in the study.
Subject agrees not to have gene therapy within the 12 weeks of their study period from initial dosing.

### Phase 2 Study Variables

### Primary Efficacy

The primary endpoint for efficacy will be a measure of functional vision as determined at week 12, using mobility testing using visual navigation challenge (VNC) course. The primary endpoint for efficacy will be a comparison between treatments groups in the mean change from randomization (baseline — last measurement prior to first dose) to week 12 in luminance levels required for successful navigation. Navigation measurements for the primary outcome on the VNC will be performed for a designated study eye.

### Secondary Efficacy

These evaluations are measured for individual eyes, i.e., each eye of the subject will be tested separately.
1. VNC evaluation at weeks 4 and 24
2. Visual field (VF) evaluations
3. Low luminance low contrast (LLLC) Best-corrected visual acuity (BCVA) (Early Treatment Diabetic Retinopathy Study [ETDRS]; letters read at 4 meters or 1 meter)
4. High luminance high contrast (HLHC) BCVA (ETDRS letters read at 4 meters or 1 meter)

All evaluations (VNC and VF) outlined above at Week 4, and if applicable also at Week 8

### Exploratory Efficacy

Evaluations can be either binocular or for each eye as routinely performed.
1. Low luminance (LL) patient-reported outcome (PRO): conducted at the subject level and not by eye at week 12.
2. EQ-5D-5L categorical results and visual analog scale conducted at the subject level at randomization, and at Week 12.
3. Spectral dominance-Optical Coherence Tomography (SD-OCT) including evaluation of the thickness of photoreceptor layer, outer/inner segment, and RPE at Screening and at Weeks 12.

Outcomes will be summarized for each cohort arm using descriptive statistics for each of these timepoints. Comparisons of each active arm to the control arm will be conducted, and a comparison using all data points from randomization through Week 12 will also be conducted. An appropriate statistical mixed effects model, including how to handle missing data from these multiple time-points, will be detailed.

### Phase 2 Study Procedures and Assessments

Dosing may be performed at the subject's home by the subject or caregiver or a home health care practitioner.

Subjects will be evaluated for safety and efficacy throughout the study. Efficacy will be assessed by mobility testing (might require travel), VF measurements, HLHC BCVA, LLLC BCVA, OCT, and PROs. Safety will be assessed by vital signs, ECG, physical examination, clinical laboratory tests, HLHC BCVA, OCT, biomicroscopic examination, IOP, fundus examination and photography, bone density measurement, hand X-ray and height measurement to assess bone development, TEAEs, and concomitant medications. Safety evaluation at each 4-week visit will include
1. Vital signs (heart rate, blood pressure, respiratory rate, body temperature, BMI)
2. Physical examination
3. ECG (with repeats as needed)
4. Clinical laboratory tests (12-hour fasting serum chemistry and lipid panel, hematology, bone chemistry, thyroid function testing, serum retinol, urinalysis and pregnancy tests in women of childbearing potential)
5. Biomicroscopic examination
6. IOP
7. Fundus examination
8. Height and Weight
10. Treatment-emergent AEs
11. Treatment-emergent SAEs
12. Concomitant medications

Clinical safety labs (chemistry including hematology, lipid panels, liver enzyme functions and urinalysis) will be reviewed by the Investigator throughout the study. Subjects will stop administration of their assigned treatment if the following abnormalities are noted while on a course of therapy:
Any SAE that is suspected to be related to treatment
Fasting ALT or AST >2.5 times the upper limit of the laboratory normal range without evidence of cholestasis (eg ALP greater than or equal to 2× ULN). In case this is noted, the test will be repeated in 4 weeks (the next visit) or sooner per Investigator's clinical judgment and planned dosing will be stopped for 4 weeks. If resumed dosing confirms the abnormal finding, there will be no dosing for 4 more weeks, and laboratory testing repeated.
Fasting triglycerides with persistent value after repeated measurement of >2.5 times the upper limit of the clinical laboratory value normal range. In case this is noted, the test will be repeated in 4 weeks (next visit) or sooner per Investigator's judgment and current dosing will be stopped for 4 weeks. If repeat dosing confirms the abnormal finding, there will be no dosing for 4 more weeks, and laboratory testing repeated.
Other potentially dose-limiting signs or symptoms as determined by the Investigator in consultation with the Sponsor's Global Medical Monitor
If there are any clinically significant safety issues as noted above, the Investigator will consult with the Sponsor's Global Medical Monitor or designee to confirm if the safety issues preclude ongoing treatment or indicate that 4 weeks of therapy should be skipped.
If it is confirmed that there are safety reasons for a subject not to continue on therapy, then subject should return for all regularly scheduled visits, and not be considered for further dosing until abnormality returns to below the specified cut-off ranges and in consultation with Global Medical Monitor.

In order to ensure patient safety from monthly courses of therapy with 9-cis-retinyl acetate, the Investigator will withdraw a subject from study treatment completely (i.e., the subject will not receive any more study treatment) in consultation with the Global Medical Monitor in the event of any of the following:
a new health condition appears that is suspected to require care or medications prohibited by the protocol
the subject has unacceptable AEs as judged by the PI in consultation with the Global Medical Monitor
the subject has any of the following clinical laboratory results:

Fasting ALT or AST >3 times the upper limit of the laboratory normal range on repeat testing that does not respond to skipping a course of dosing (i.e. remains >3 times upper limit of laboratory normal for more than 4 weeks off therapy).

Fasting triglycerides >3 times the upper limit of the laboratory normal range on repeat testing that does not respond to skipping a course of dosing (i.e. remains >3 times upper limit of laboratory normal for more than 4 weeks off therapy).

Development of thyroid abnormality that meets CTC grade 2 criteria (symptomatic and requires medical intervention) and does not respond to treatment (at the Investigator's discretion) within 4 weeks

Positive pregnancy test
the subject has a clinically significant hypersensitivity reaction to study treatment as judged by the Investigator in consultation with the Global Medical Monitor
the subject exhibits clinically significant signs of hypervitaminosis A syndrome, particularly if symptoms occur that indicate pseudotumor cerebri (an increase in intracranial pressure)
there is a prolongation of the QTcB interval, such that:

it is >500 msec when the measurement obtained is averaged from duplicate ECGs, or

in the Investigator's judgment an otherwise marked prolongation may indicate that the subject's safety is at issue
it is in the subject's best interest according to the Investigator's clinical judgment

Withdrawal from treatment for the reasons noted above will be reviewed in consultation with the Sponsor's Global Medical Monitor. Subjects should still be brought back for all visits and evaluated
Blood samples for PK analysis will be collected per schedule

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of treating a subject having a visual disorder comprising administering daily to the subject, a dosage of about 0.1 mg to 20 mg of 9-cis-retinyl acetate.

2. The method of claim 1, wherein the dosage of 9-cis-retinyl acetate is selected from the group consisting of from about 0.5 mg to about 10 mg and from about 0.5 mg to about 5 mg.

3. (canceled)

4. The method of claim 1, wherein the dosage of 9-cis-retinyl acetate is selected from the group consisting of about 0.5 mg, about 1 mg, about 1.5 mg, about 2 mg, about 3 mg, about 4 mg, and about 5 mg.

5-10. (canceled)

11. The method of claim 1, wherein 9-cis-retinyl acetate is administered once daily.

12. (canceled)

13. The method of claim 1, wherein the 9-cis-retinyl acetate is administered for a period selected from the group consisting of at least 30 days, at least 60 days, and at least 90 days.

14-15. (canceled)

16. A method of treating a subject having a visual disorder, the method comprising administering to the subject an effective amount of 9-cis-retinyl acetate once daily, wherein the effective amount of 9-cis-retinyl acetate maintains a trough circulating blood concentration of 9-cis-retinol selected from the group consisting of at least 2 nM, at least 3 nM, and at least 4 nM.

17-18. (canceled)

19. The method of claim 16, wherein the effective amount of 9-cis-retinyl acetate maintains a circulating blood concentration of 9-cis-retinol selected from the group consisting of from about 2 to about 20 nM and from about 3 to about 10 nM.

20. (canceled)

21. The method of claim 16, wherein the Cₘₐₓ of 9-cis-retinol observed after once daily dosing of 9-cis-retinyl acetate does not exceed a level selected from the group consisting of 15 nM and 20 nM.

22. (canceled)

23. The method of claim 1, wherein the subject has a mutation in the LRAT gene.

24. The method of claim 1, wherein the subject has a mutation in the RPE65 gene.

25. The method of claim 1, wherein the visual disorder is an endogenous retinoid deficiency.

26. The method of claim 1, wherein the visual disorder is selected from the group consisting of Leber congenital amaurosis (LCA), retinitis pigmentosa (RP), autosomal recessive retinitis pigmentosa (arRP), age-related retinyl dysfunction, nyctalopia, retinitis punctata albesciens, congenital stationary night blindness (CSNB), fundus albipunctatus, age-related macular degeneration (AMD), and Stargardt disease.

27. The method of claim 1, wherein the visual disorder is associated with rod-mediated dark adaptation after light exposure, an impairment in night vision, an impairment in contrast sensitivity, an impairment in visual field, or an impairment in visual acuity.

28. The method of claim 1, wherein administration of 9-cis-retinyl acetate improves the visual function of the subject as determined by an assessment method selected from the group consisting of a visual navigational challenge (VNC) test, a visual field (VF) evaluation, a low luminance low contrast (LLLC) best corrected visual acuity (BCVA) test, a high luminance high contrast (HLHC) BCVA test, an optical coherence tomography (OCT) test, and a patient reported outcome (PRO) quality of life (QoL) questionnaire, and a low luminance (LL) questionnaire.

29. The method of claim 28, wherein the assessment method is a VNC test, the VNC test provides a VNC score, and administration of 9-cis-retinyl acetate results in an improvement of the VNC score of the subject relative to a baseline score of the subject prior to administration of 9-cis-retinyl acetate, and wherein the improvement of the VNC score is selected from the group consisting of 1, 2, 3, and 4 luminance level.

30-34. (canceled)

35. The method of claim 1, wherein 9-cis-retinyl acetate is administered orally.

36-44. (canceled)

45. A method of treating a subject having a visual disorder comprising administering daily to the subject, a dosage of about 0.1 mg to 20 mg of 9-cis-retinyl acetate; wherein the dosage comprises an effective amount of 9-cis-retinyl acetate to be administered once daily; and the effective amount of 9-cis-retinyl acetate maintains a trough circulating blood concentration of 9-cis-retinol selected from the group consisting of at least 2 nM, at least 3 nM, and at least 4 nM.

46. The method of claim 45, wherein the Cₘₐₓ of 9-cis-retinol observed after once daily dosing of 9-cis-retinyl acetate does not exceed a level selected from the group consisting of 15 nM and 20 nM.

47. The method of claim 46; wherein administration of 9-cis-retinyl acetate improves the visual function of the subject as determined by a visual navigational challenge (VNC) test; wherein the VNC test provides a VNC score, and administration of 9-cis-retinyl acetate results in an improvement of the VNC score of the subject relative to a baseline score of the subject prior to administration of 9-cis-retinyl acetate; and wherein the improvement of the VNC score is selected from the group consisting of 1, 2, 3, and 4 luminance level.

48. The method of claim 47, wherein the daily dosage is administered orally.
